(19) United States
(12) Patent Application Publication
(10) Pub. No.: US 2011/0071229 A1
(43) Pub. Date: Mar. 24, 2011

(54) Synthetic Gas Recycle Apparatus and Methods

(75) Inventors: Mark K. Robertson, Broomfield, CO (US); Gwo-Jang Abraham Liou, Sugar Land, TX (US)

(73) Assignee: SYNTHESIS ENERGY SYSTEMS, INC., Houston, TX (US)

(21) Appl. No.: 12/563,800

(22) Filed: Sep. 21, 2009

(51) Int. Cl. C07C 1/04; C07C 27/06; C07C 29/00

(52) U.S. Cl. 518/702; 422/187

(57) ABSTRACT

Processes and apparatuses for synthesizing a hydrocarbon by reacting a carbon-containing feedstock with an oxygen-containing gas stream having a greater molar ratio of oxygen than the feedstock to produce a first synthetic gas comprising carbon monoxide and hydrogen, reacting the first synthetic gas in a shift reaction zone to produce a shifted synthetic gas, optionally but preferably blending a recycled synthetic gas with the shifted synthetic gas to produce a second synthetic gas, reacting the second synthetic gas with a catalyst in a synthesis reaction zone to produce hydrocarbon(s) and a purge gas, passing the purge gas through a separation zone to produce a hydrogen-containing permeate stream having an increased hydrogen molar ratio compared to the purge gas and a methane-containing non-permeate stream having an increased methane molar ratio compared to the purge gas, and reacting the methane-containing non-permeate stream in a reformation zone to produce the recycled synthetic gas.

### FIELD OF THE INVENTION

The invention relates to processes and apparatus for gasifying hydrocarbon-containing material into a synthesis gas, and in other embodiments, for generating hydrocarbons therefrom. In particular, a first synthetic gas including hydrogen and carbon monoxide can be reacted in a shift reaction zone to produce a shifted synthetic gas, and preferably blending a recycled synthetic gas with the shifted synthetic gas to produce a second synthetic gas.

### BACKGROUND

Several methods for gasifying carbon-containing material into raw synthesis gas (“syngas”) are known in the industry. Carbon-containing fuel, such as coal or oil, can be gasified in a gasification reactor to produce raw syngas.

It is also known to partially shift raw syngas so as to provide a desired H₂/CO ratio for downstream hydrocarbon synthesis, such as methanol synthesis. The shift reactor can also convert carbonyl sulfide (COS) in the raw syngas to H₂S and CO₂, allowing for the H₂S and CO₂ to be removed by an acid gas removal system.

The shifted syngas can then be converted to hydrocarbons in a synthesis reactor in the presence of a transition metal catalyst.

A membrane unit may be used to separate H₂ from the purge gas generated by the synthesis reactor. Part or all of this H₂ may be returned to the synthesis reactor at various reaction stages to optimize hydrocarbon yields.

Electric power may also be generated by this process through integrated power systems. For integrated power applications, part or all of the unreacted syngas from the synthesis reactor is fired, or combusted, as fuel, typically in a gas turbine system that drives a generator to produce electric power. Hot turbine exhaust can then be passed to a heat recovery system to produce high pressure steam. This stream may be expanded through another steam turbine to drive another electric generator to produce additional power. This is not always desirable and can often result in excess unreacted synthesis gas. Thus, it is desired to provide an improved apparatus and methods that recycle excess unreacted synthesis gas to increase syngas efficiencies. The present invention described below advantageously achieves this goal.

### SUMMARY OF THE INVENTION

The invention encompasses processes for synthesizing a hydrocarbon that includes reacting a carbon-containing feedstock with an oxygen-containing gas having a greater molar ratio of oxygen than the feedstock to produce a first synthetic gas including carbon monoxide and hydrogen having a ratio of carbon monoxide to hydrogen; reacting the first synthetic gas in a shift reaction zone to produce a second synthetic gas; reacting the second synthetic gas in association with a catalyst in a synthesis reaction zone to produce one or more hydrocarbons and a purge gas; passing the purge gas through a separation zone to produce a hydrogen-containing permeate stream having an increased hydrogen molar ratio compared to the purge gas and a methane-containing non-permeate stream having an increased methane molar ratio compared to the purge gas; reacting the methane-containing non-permeate stream in a reformation zone to produce a recycled synthetic gas; and optionally but preferably blending the recycled synthetic gas with the second synthetic gas. In another embodiment, the invention encompasses a process for synthesizing a hydrocarbon that includes reacting a carbon-containing feedstock with an oxygen-containing gas stream having a greater molar ratio of oxygen than the feedstock to produce a first synthetic gas comprising carbon monoxide and hydrogen having a ratio of carbon monoxide to hydrogen, reacting the first synthetic gas in a shift reaction zone to produce a shifted synthetic gas, optionally but preferably blending a recycled synthetic gas with the shifted synthetic gas to produce a second synthetic gas, reacting the second synthetic gas in association with a catalyst in a synthesis reaction zone to produce one or more hydrocarbons and a purge gas, passing the purge gas through a separation zone to produce a hydrogen-containing permeate stream having an increased hydrogen molar ratio compared to the purge gas and a methane-containing non-permeate stream having an increased methane molar ratio compared to the purge gas, and reacting the methane-containing non-permeate stream in a reformation zone to produce the recycled synthetic gas.

In one embodiment, the process includes blending the recycled synthetic gas with the second synthetic gas to increase the yield of one or more hydrocarbons produced in the synthesis reaction zone. In another embodiment, the process further includes increasing the ratio of hydrogen to carbon monoxide in the synthesis reaction zone. In yet another embodiment, the process further includes separating ambient air in an air separation zone to increase the oxygen ratio in the oxygen-containing gas compared to air. In a further embodiment, the process further includes removing one or more sulfur-containing compounds from the second synthetic gas. In a preferred embodiment, the process further includes recovering an amount of sulfur from the one or more sulfur-containing compounds in a sulfur recovery zone.

In a further embodiment, the process further includes returning the hydrogen-containing permeate stream to the synthesis reaction zone. In one preferred embodiment, the carbon-containing feedstock includes coal. In another preferred embodiment, the reformation zone includes a steam methane reformer.

The invention further encompasses a process for synthesizing methanol that includes reacting a feedstock including coal with an oxygen-containing gas to produce a first synthetic gas including carbon monoxide and hydrogen having a ratio of carbon monoxide to hydrogen; feeding at least a portion of the first synthetic gas to a shift reaction zone; reacting the first synthetic gas in the shift reaction zone to produce a second synthetic gas; reacting the second synthetic gas over a catalyst in a synthesis reaction zone to produce methanol and a purge gas; passing the purge gas through a separation zone to produce a hydrogen-containing permeate stream having an increased hydrogen molar ratio compared to the purge gas and a methane-containing non-permeate stream having an increased methane molar ratio compared to the purge gas; reacting the methane rich non-permeate stream in a steam methane reformation zone to produce a recycled synthetic gas; and optionally but preferably blending the recycled synthetic gas stream and the second synthetic gas.

The invention further encompasses a hydrocarbon-generating apparatus including a first reactor adapted to react a carbon-containing feedstock with an oxygen-containing gas stream having a greater molar ratio of oxygen than the feedstock to produce a first synthetic gas including carbon monoxide and hydrogen having a ratio of carbon monoxide to hydrogen; a shift reactor adapted to react carbon monoxide and water in the first synthetic gas to form carbon dioxide and hydrogen to produce a second synthetic gas having a ratio of carbon monoxide to hydrogen less than that of the first synthetic gas; a synthesis reactor adapted to react a combination of the second synthetic gas and a recycled synthetic gas in association with a catalyst to produce one or more hydrocarbons and a purge gas; a separation unit adapted to separate the purge gas into a hydrogen-containing permeate stream having an increased hydrogen molar ratio compared to the purge gas and a methane-containing non-permeate stream having an increased methane molar ratio compared to the purge gas; and a reformation reactor adapted to react the methane-containing non-permeate stream to produce the recycled synthetic gas stream.

In one embodiment, the shift reaction is adapted to increase the ratio of hydrogen to carbon monoxide produced upon reaction of the first synthetic gas. In another embodiment, the recycled synthetic gas is combined with the second synthetic gas to increase the ratio of hydrogen to carbon monoxide in the synthesis reactor. In a further embodiment, the apparatus further includes an air separation unit adapted to separate oxygen from ambient air to increase the oxygen content in the oxygen-containing gas stream. In yet a further embodiment, the apparatus includes an acid gas removal unit adapted to remove one or more sulfur-containing compounds from the second synthetic gas. In a preferred embodiment, a sulfur recovery unit is adapted to recover an amount of sulfur from the one or more sulfur-containing compounds. In a further embodiment, the apparatus further includes a return stream that introduces the hydrogen-containing permeate into the synthesis reaction zone. It should be understood that each of the above-noted embodiments applies equally to any aspect of the invention as described above or any others disclosed below.

### BRIEF DESCRIPTION OF THE DRAWINGS

Further features and advantages of the invention can be ascertained from the following detailed description that is provided in connection with the drawing(s) described below:

FIG. 1A describes a basic embodiment of the present invention, integrating coal gasification using a shift reactor with a synthesis gas recycle stream; and

FIG. 1B illustrates one preferred embodiment of the present invention, integrating methanol synthesis with a membrane separation unit and a stream reformer.

### DETAILED DESCRIPTION OF PREFERRED EMBODIMENTS

According to the invention, unreacted synthesis gas from a purge gas stream may be used as a feed to a separation zone, such as a membrane separation zone, from which a non-permeate methane-increased stream may then be fed into a steam reformer. This steam reformer can advantageously operate to convert a portion of the methane generated from a preferred carbon-containing feedstock, which was separated from a purge gas stream by the separation zone (e.g., membrane separation unit), into a syngas recycle stream that can then be fed back into a shift reactor bypass stream to increase syngas production efficiency. The invention disclosed in the following specification and defined in the appended claims provides for a gasification apparatus including such a steam reformer and syngas recycle stream. In discussing the invention below, while certain specific equipment is used to describe an apparatus or a system according to the invention, with reference to the processes according to the invention, it should be understood that the processes are not limited to these specific equipment references and that a similar zone achieving a similar function can be used. For example, a shift reactor and shift reactor bypass stream may be used according to the apparatus, however, the process of the invention would involve a shift reaction zone.

In the present invention, carbon-containing fuel is first obtained, and can be prepared in advance of, then fed to a gasification reactor. Carbon-containing fuel includes any solid, liquid or gaseous combustible organic feedstock used in a high-temperature gasification process to produce synthesis gas (“syngas”). The carbon-containing fuel feedstock provides a source of hydrogen and carbon for the gasification reaction. The carbon-containing fuel can be in a liquid, solid or gaseous state, or in a solid-liquid combination; for example, it can be in the form of a gel, or a fluidized state including without limitation as a suspension, emulsion, or slurry. In a preferred embodiment of the gasification processes, the carbon-containing fuel includes crushed coal fed to the gasification reactor, and more preferably a fuel that is entirely crushed solids, including but not limited to granules, pellets, powders, or grains of solids. In one embodiment, this is coal substantially free of impurities. The carbon-containing fuel is preferably carried by a CO₂-rich carrier gas. Any size crushed solids may be used, preferably provided that the crushed solids are sized and dimensioned so as to be carried by the carrier gas without any substantial precipitation from the carrier gas. Gasification of carbonaceous feedstocks tends to produce raw syngas that is carbon or carbon monoxide rich and hydrogen deficient, a concern that the present invention can advantageously address, as discussed herein. In one embodiment, it is preferred that the feedstock be substantially free, or entirely free, of natural gas.

The carbon-containing fuel is typically reacted in the gasification reactor in an oxygen deficient environment with a reactive oxygen-containing gas, such as air, enriched air, or pure oxygen, and optionally but preferably, a temperature moderator, including, without limitation, water or steam, to obtain a first syngas, sometimes called “raw syngas.”

In a preferred embodiment, an air separation unit (“ASU”) is used to separate ambient air into separate streams of a predominantly oxygen-containing gas and a predominantly nitrogen-containing gas. Preferably, the oxygen-containing gas is at least about 60 mole percent oxygen, more preferably at least about 80 mole percent oxygen. In most preferred embodiments, the oxygen-containing gas is at least substantially pure oxygen gas. As to the ASU, the term “substantially pure oxygen gas” is oxygen relative only to nitrogen and other gases, rather than the total weight of the syngas. In one embodiment, all of the oxygen-containing gas from the ASU is introduced into the gasification reactor as the oxygen-containing gas in desired amounts based on the guidance herein. In another embodiment, a portion of the substantially pure oxygen-containing gas from the ASU is introduced into an acid gas removal system, for example, to remove at least a portion of any sulfur-containing compounds present using a Claus-type or other type of sulfur recovery unit (SRU). In yet another embodiment, a portion of the substantially pure oxygen-containing gas from the ASU is introduced into a reformer. The reformer may be an autothermal reformer or a steam reformer.

The type of temperature moderator used to control the temperature in the gasification reactor depends in part on the carbon/hydrogen ratio of the feedstock and the oxygen content of the oxygen-containing gas. Preferred temperature moderators include water or steam, preferably water or steam that is substantially pure to minimize or avoid fouling of the apparatus of the invention from particles present therein. Other potential temperature moderators include the CO₂-rich carrier gas or a N₂-rich gas, the “rich” being in reference to the typical amounts of these components present in ambient air. Steam, e.g., may be used as a temperature moderator by mixing it with either the feedstock fuel or the oxygen-containing gas stream. Alternatively, a temperature moderator, such as steam, may be introduced into the gasification reactor by way of a direct or indirect conduit in the gasification reactor burner. A temperature moderator is preferably used in association with a liquid fuel where substantially pure oxygen is the oxygen-containing gas.

Any suitable gasification reactor may be used based on the guidance to one of ordinary skill in the art provided herein as to the invention. In one embodiment, the gasification reactor is a fixed bed (up-draft) type. In another embodiment, the gasification reactor is a co-current fixed bed (down-draft) gasification reactor type in which the oxygen-containing gas flows in a co-current direction with the carbon-containing fuel. In this type of gasification reactor, heat is preferably transferred into the upper part of the gasification reactor by combusting a portion of the carbon-containing fuel or by transfer from external heat sources. One such external heat source can include combustion of one or more hydrocarbons produced in excess from the synthesis reactor.

In a preferred embodiment, however, the gasification reactor is a counter-current type or both counter-current and fixed bed (up-draft) type consisting of a bed of carbonaceous fuel (e.g., coal), through which the oxygen-containing gas flows in counter-current relation to the fuel. The ash from a counter-current fixed bed type of gasification reactor may be removed dry or as a slag according to techniques available to those of ordinary skill in the art.

In another preferred embodiment, the gasification reactor is a fluidized bed type, in which the fuel (e.g., coal) is fluidized in the oxygen-containing gas. Fluidized bed reactors tend to be most useful in connection with feedstocks that tend to create corrosive ash, which typically damages the walls of fixed bed type gasification reactors. Fluidized bed reactors also tend to produce higher amounts of methane, relative to other types of gasification reactors. The ash from a fluidized bed type of gasification reactor can be removed as dry ash or as defluidized agglomerates, or by any other available conventional technique suited to the equipment of the invention. Reaction temperatures are typically relatively lower in fluidized gasification reactors, so highly-reactive fuels, such as low-grade coal, are particularly suitable as feedstocks for such equipment. Fluidized bed gasification reactors that generate agglomerates tend to operate at slightly higher temperatures, suitable for high-rank coal feedstocks. Recycling or subsequent combustion of unreacted feedstock from fluidized bed gasification reactors can be employed to increase syngas conversion rates.

The present invention is particularly relevant where a low temperature, non-slagging counter-current fixed bed or a fluidized bed gasification reactor produces syngas useful for downstream methanol synthesis.

In a conventional gasification/synthesis process, the first syngas exiting a gasification reactor will typically consist primarily of hydrogen (H₂) and carbon monoxide (CO), along with smaller quantities of water (H₂O), carbon dioxide (CO₂), and possibly carbonyl sulfide (COS) and hydrogen sulfide (H₂S).

The first syngas produced by the gasification of carbon-containing feedstocks is generally carbon (e.g., CO) rich and hydrogen (H₂) deficient. To produce an optimal H₂/CO ratio for downstream synthesis processes, a shift reactor can be used to generate additional H₂ by a gas-shift reaction, during which carbon monoxide preferably reacts with water to form carbon dioxide and hydrogen according to the chemical equation:

CO+H₂O→CO₂+H₂

The gas-shift reaction typically occurs by introducing water to the first syngas stream, or using the water already contained in the first syngas stream and reacting the mixture adiabatically over a shift catalyst. The shift reactor converts water and carbon monoxide in the first syngas or in any added feedstock to a second syngas that includes hydrogen and carbon dioxide. Some advantages of a gas-shift reaction, as performed by the shift reactor, can include an increase in the amount of hydrogen and a reduction in the amount of carbon monoxide in the second syngas. In a preferred embodiment, the gas shift reaction is the sour shift type, where the shift reactor is located upstream of the acid gas removal system and the shift reaction is exothermic. This heat can then be used elsewhere in the process and apparatus described herein, such as to help maintain or increase the temperature of another portion of the inventive process or one or more other associated processes.

The shift catalyst can be any suitable catalyst available to those of ordinary skill in the art, but is preferably one or more Group VIII metals. Preferably, the catalyst includes a metal associated with a heat-resistant support. For example, without limitation, the shift catalyst may be ceramic-supported, such as random-packed ceramic supported catalyst pieces. The catalyst may be formed as beads or micro- or nano-particles, or a monolithic catalyst having through-passages generally parallel to the direction of the raw syngas flow, or any combination thereof.

The gas-shift reaction is reversible, with lower temperatures favoring hydrogen and carbon dioxide formation. The catalyzed reaction rate is typically, however, slower at lower temperatures. Therefore, it is often advantageous to have higher temperature and low temperature shift reactions in sequence. High temperature shift catalysts typically include iron oxide, preferably combined with a relatively lesser amount of chromium oxide. Low temperature shift catalysts typically include one or more copper oxides preferably supported on zinc oxide, alumina, or a combination thereof. In a preferred embodiment, the design and operation of the shift reactor causes as minimal a pressure drop as feasible. Thus, the pressure of the syngas can be preserved for downstream hydrocarbon synthesis processes without using additional energy to increase the syngas pressure.

In a preferred embodiment, the first syngas is typically passed through a low temperature gas cooling system in the shift reaction zone, in which the syngas is cooled and heat is recovered from syngas, typically generating steam or heat that can optionally but preferably be used elsewhere in the process of the invention. The low temperature gas cooling system also helps condense and remove at least substantially all of the water from the first syngas.

By passing though the shift reaction zone, the first syngas is converted to a shifted syngas. The shifted syngas is then blended with a recycled syngas stream, as later described, to produce a second syngas. The second syngas is preferably treated to reduce the amount of acid gases, particularly CO₂, COS, and H₂S, to make the syngas substantially pure with respect to the acid gases before the syngas is used in downstream processes. The removal of sulfur-containing compounds helps to inhibit or prevent catalyst poisoning downstream, while the reduction of CO₂ helps to provide an increased and more optimal hydrogen to carbon ratio for downstream synthesis processes. A number of acid gas removal systems are commercially available to treat syngas, and any of these are suitable if selected by one of ordinary skill in the art to be operable as part of the invention based on the guidance herein. Selection of an acid gas removal system can depend on various factors, including the degree of removal desired, the energy and sizing requirements of such a system, and by the desired operating pressure of the acid gas removal system.

As part of the acid gas removal system, an acid gas removal solvent can be used to absorb a portion of the acid gas from the second syngas, typically at a relatively high pressure, for example without limitation from about 2100 KPa to 7000 KPa. After absorption, the removal solvent containing the acid gases is typically let down in pressure, steam stripped, or both, to release and recover at least substantially all of the acid gases. The acid gas removal solvent may operate selectively, allowing recovery of H₂S and CO₂ as separate streams, so that the H₂S stream can be sent to a sulfur recovery unit for conversion to elemental sulfur while the CO₂ can be compressed and sequestered, or used for enhanced product yields in the gasification reactor. Alternatively, the acid gas removal solvent may be selected simply to neutralize or to strip substantially all acid gas from the second syngas.

The syngas exiting the acid gas removal system enters a synthesis reactor to be converted to hydrocarbons, typically in the presence of a transition metal catalyst that is known to catalyze the conversion of CO and H₂ to certain desired hydrocarbons. Suitable catalysts include cobalt, iron, or both, on an alumina support. Other Group VIII metals, including without limitation ruthenium and osmium, or any combination with the above-noted materials, are also usable catalysts. Other metals that are potential catalysts include rhenium, molybdenum, and chromium. Any of these catalyst materials in any combination may be used as suitable when one of ordinary skill in the art considers the other factors in preparing an apparatus or process of the invention. The activities of these catalysts are preferably enhanced by adding other metals, such as copper, cerium, rhenium, manganese, platinum, iridium, rhodium, molybdenum, tungsten, ruthenium or zirconium, or any combination thereof. The types and amounts of reaction hydrocarbons obtained via synthesis over one of these catalysts varies based upon many conditions, such as reactor type, process conditions, and type of catalyst used.

In a preferred embodiment, the reactor type, process conditions, and catalyst are chosen to selectively generate methanol as a product of the synthesis reaction. The general chemistry of a methanol synthesis reaction is as follows:

CO+2H₂→CH₃OH

In this preferred embodiment, carbon monoxide and hydrogen react on a catalyst to produce methanol. The catalyst preferably includes a mixture of copper, zinc oxide, and alumina. At about 5000 KPa to 10000 KPa and a temperature of about 225° C. to 275° C., preferably about 240° C. to 260° C., and in an exemplary embodiment, about 250° C., such a catalyst is capable of catalyzing the production of methanol from carbon monoxide and hydrogen with high selectivity.

In certain embodiments, the synthesis reactor is an entrained bed reactor, fixed-fluidized bed reactor, or slurry bubble column reactor. In a preferred embodiment, the synthesis reactor is a tubular steam-raising fixed bed reactor.

The products from the hydrocarbon synthesis reactor can be divided into streams of purge gas, product gas, and wastewater. Methanol is recovered from the product gas preferably by cooling to below the dew point of the methanol and separating off the product as a liquid. The hydrocarbon synthesis process is preferably operated in a loop, with a portion of the unreacted syngas recycled back through the synthesis reactor. The amount of side reaction products and water in the various product streams depends in part on the type of reactor, catalyst employed and process conditions. The purge gas from a synthesis reactor typically includes water vapor, CO₂, CH₄, N₂, unreacted syngas (H₂ and CO), and gaseous hydrocarbon products.

In a preferred embodiment, a separation zone, for example without limitation including a membrane separation unit is used to separate H₂ gas from the purge gas. The membrane separation unit typically employs a membrane that is selectively permeable to H₂ to obtain a relatively hydrogen-rich permeate compared to the non-permeate. A portion of this H₂ rich permeate may be sent back to the synthesis reactor at various stages to optimize yields of the reaction in the synthesis reactor, or synthesis reaction zone, according to the invention. The non-permeate stream from the membrane separation unit typically includes methane, carbon monoxide, and other components and can be further processed or directly used as fuel for any heating needed as described herein. In the present invention, the non-permeate stream is used as a feed stream to a reformer.

A recycle syngas stream may then be generated in the reformer from the non-permeate stream. The reformer equipment may be of any type available to one of ordinary skill in the art. For example, in a steam methane reformer (“SMR”), at moderate pressures of about 1000 KPa to 2000 KPa and a temperature of about 800° C. to 900° C., preferably about 825° C. to 875° C., and in an exemplary embodiment at about 850° C., the methane in the non-permeate stream will react with steam on a nickel catalyst to produce syngas according to the chemical equation:

CH₄+H₂O→CO+3H₂

The methane in the non-permeate stream can also undergo partial oxidation with molecular oxygen to produce syngas according to the chemical equation:

2CH₄+O₂→2CO+4H₂

When these two chemical processes are combined in a single reformer, the reforming process is typically referred to as autothermal reforming.

The purge gas is preferably recycled by directing it to be reformed to generate a relatively hydrogen-rich recycle syngas stream that can be advantageously blended with the relatively carbon-rich syngas in the shift reaction zone. In a preferred embodiment, the relatively hydrogen-rich recycle syngas stream reenters the process in the shift reaction zone and comprises a portion of the second syngas. With this syngas recycle stream, less shifting of the CO in the first syngas will be required to achieve a desired H₂/CO ratio for the downstream synthesis reactor. The recycle syngas stream results in more CO being available for conversion into the desired hydrocarbon product.

The recycle syngas stream can significantly increase conversion of the gasification feedstock into the desired synthesis product(s). The recycle syngas stream is useful when the permeate stream reformed to generate the recycle syngas stream contains significant quantities of light hydrocarbons, such as when the purge gas from a methanol unit synthesis reactor has a high methane content. By providing additional H₂ feedstock for the synthesis, this can increase the methanol production efficiency by at least about 1% to 50%, and in varying embodiments by at least about 5%, at least about 10%, at least about 15%, at least about 20%, and at least about 30% compared to the methanol yield from a conventional gasification/synthesis, particularly using a hydrocarbon feedstock that includes coal or is exclusively coal.

The synthesis reactor also can produce as byproducts one or more of the following including: carbon dioxide, water, and oxygenated components, including acids such as acetic acid, formic acid, propionic acid; alcohols such as, ethyl alcohol, propyl alcohol, and longer chained alcohols; aldehydes, ketones and esters. Typically, these oxygenated components make up a small fraction of the methanol synthesis reaction products and, because of their water-soluble nature, can be found in the wastewater stream from the synthesis reactor. The wastewater stream is usually sent to a water treatment system to remove substantially all of the undesired components in potable water, and these removed “undesirable” components can be collected for recycling, used in related on-site chemical processing, or sold as a feedstock to others.

Removal of the wastewater stream and the purge gas stream leaves the desired hydrocarbon product stream. The hydrocarbon product is preferably liquid methanol and is generally processed in a downstream storage and loading system, although it could be loaded directly.

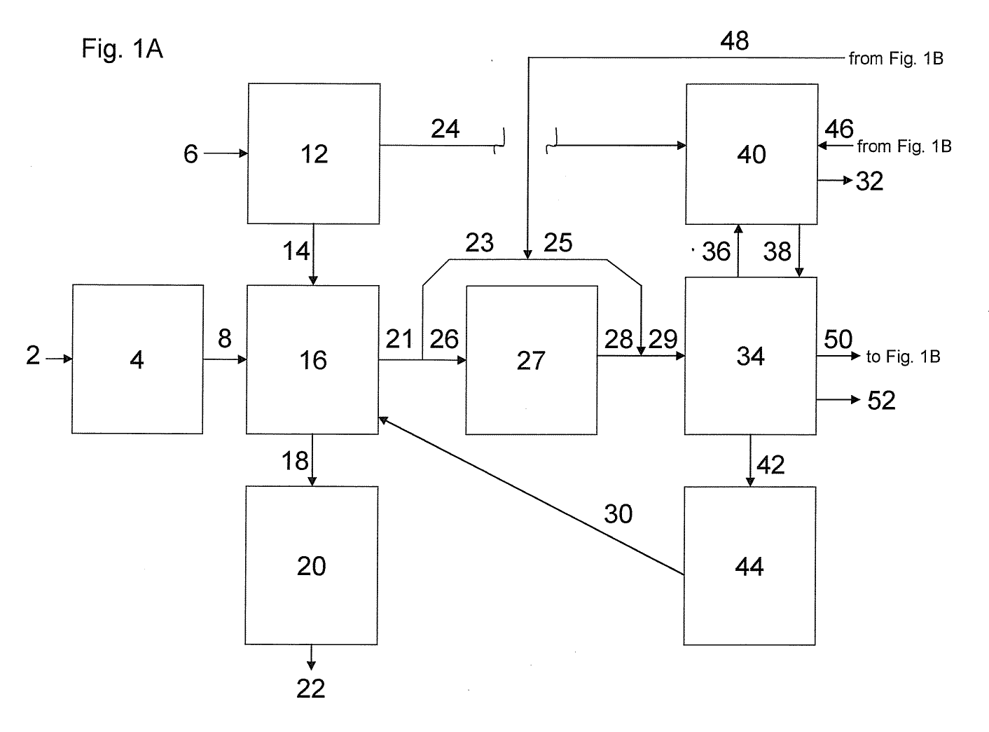

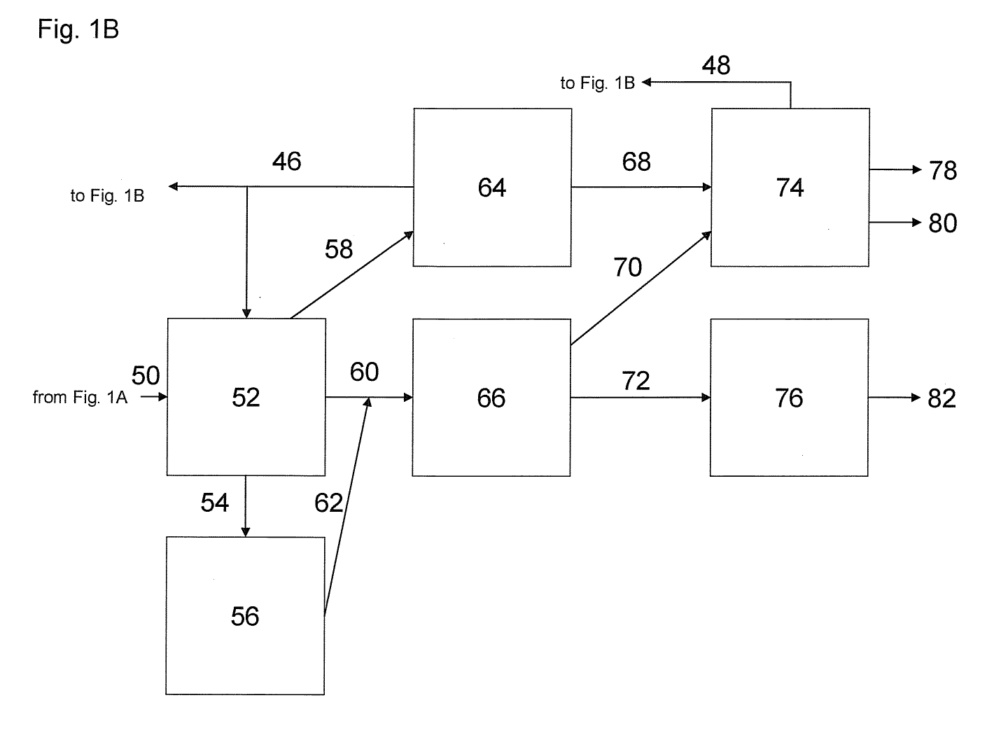

FIG. 1A shows an embodiment of the present invention. Carbon-containing fuel 2 goes through coal handling and preparation 4, becoming feedstock stream 8. In a preferred embodiment, air 6 enters air separation unit 12, producing oxygen rich gas streams 14 and 24. Feedstock stream 8 and oxygen rich gas stream 14 enter gasification reactor 16. Gasification reactor produces raw syngas 21, and ash 18. While it should be understood that any suitable scaling can be implemented, exemplary amounts are provided in this example by way of providing guidance and illustrating relative amounts in different portions of the process and apparatus. In a preferred embodiment, the flow rate of the raw syngas 21 can be about 100 to 150 MMSCFD, for example about 125.7 MMSCFD. Ash 18 goes through ash handling 20 to ash exit stream 22. A portion of raw syngas 26 enters shift reactor 27, which produces shifted syngas 28. In the shift reactor 27, CO is converted in the presence of water to H₂ and CO₂ to optimize the H₂/CO ratio in the shifted syngas 28. In a preferred embodiment, this portion can be about 55% to 85%, more preferably about 60% to 75%, and in an exemplary amount is about 68%, of the total raw syngas 21 produced by the gasification reactor 16. A second portion of raw syngas 23 bypasses the shift reactor in this depicted embodiment, and is blended with a syngas recycle stream 48 to form blended syngas stream 25. Blended syngas stream 25 is then combined with shifted syngas 28 to form second syngas 29, which is fed to acid gas removal unit 34. Oxygen-rich stream 24 is also fed to sulfur recovery unit 40. Acid gas removal unit 34 produces acid gas 36, syngas 50 and carbon dioxide streams 52 and 42. In a preferred embodiment, the flow rate of syngas 50 can be about 140 MMSCFD to 200 MMSCFD, an in an exemplary amount is about 165.6 MMSCFD, and the flow rate of carbon dioxide stream 52 can be about 40 to 70 MMSCFD, and in an exemplary amount is about 54.7 MMSCFD. Acid gas 36 enters sulfur recovery unit 40, where sulfur 32 in the acid gas is removed. Sulfur recovery unit 40 produces sulfur 32 and tail gas 38, which is recycled back to acid gas removal unit 34. Carbon dioxide stream 42 is passed through a compressor 44 and recycled in a carbon dioxide recycle stream 30 back to gasification reactor 16.

FIG. 1B shows an embodiment of the present invention, and is a continuation of the embodiment illustrated in FIG. 1A. Thus, for convenience purposes, the same line numbers are used as in FIG. 1B where applicable. Syngas 50 is sent to the synthesis reactor 52. Synthesis reactor 52 produces the desired crude hydrocarbons 54 and desired crude hydrocarbons 60 and a purge gas 58. Hydrocarbons 60 are sent to distillation unit 66. Hydrocarbons 54 are optionally sent to storage 56, and can be stored for streaming along line 62 to distillation unit 66 at a later time. Distillation unit 66 produces fuel gas 70 and hydrocarbon product 72.

Purge gas stream 58 is passed through separation zone 64 (e.g., a membrane separation unit), where a hydrogen rich stream 46 is produced as a permeate and a methane rich stream 68 is produced as a non-permeate. Hydrogen rich stream 46 is recycled back to the sulfur recovery unit 40. The methane rich stream 68 is fed to a reformer 74, which is optionally partially fired by fuel gas 70. Reformer 74 produced syngas recycle stream 48, flue gas 78, and steam 80. Syngas recycle steam 48 is recycled back to be blended with the second portion of raw syngas 23, which can ultimately increase the H₂/CO ratio in the syngas 50. In a preferred embodiment, the flow rate of syngas recycle stream 48 can be about 30 to 55 MMSCFD, preferably about 35 to 45 MMSCFD, an in an exemplary embodiment is about 41.5 MMSCFD.

Steam 80 can be exported for use as a heat source in other processes, or can be expanded to produce additional power. Hydrocarbon product 72 is sent to storage and loading unit 76 before exiting the process as final product stream 82. Is a preferred embodiment, the final product stream 82 includes methanol at a flow rate of about 1000 STPD to 3000 STPD, and in an exemplary embodiment is about 2210 STPD.

While different steps, processes, and procedures are described as appearing as distinct acts, it is understood that the steps, process, and procedures could also be performed in different orders, simultaneously, or sequentially. Additionally, the steps, processes, and procedures could be merged into one or more steps, processes, or procedures.

The term “about,” as used herein, should generally be understood to refer to both numbers in a range of numerals such that “about 5 to 20” should be understood to mean “about 5 to about 20.” Moreover, all numerical ranges herein should be understood to include each whole integer within the range. Unless otherwise specified, all percentages herein refer to weight percent instead of volume percent.

The teams “substantially,” or “substantially pure,” as used herein, refers to at least about 90 weight percent, typically at least about 95 weight percent, and preferably can refer to at least about 98 weight percent, and more preferably at least about 99 weight percent of the desired component, e.g., methane, based on the total weight of the syngas. In one more preferred embodiments, the term means at least about 95.5 weight percent, preferably at least about 99.9 weight percent, and more preferably at least about 99.99 weight percent, of the desired component. The term “substantially free” refers to the corresponding amounts of 100 percent less the relevant amount above, e.g., less than about 10 weight percent, typically less than about 5 weight percent, and preferably less than about 2 weight percent, etc.

### EXAMPLE

The following example is presented to further illustrate various aspects of the present invention, and is not intended to limit the scope of the invention.

### Gasification of Coal to Generate Methanol

An apparatus was prepared according to the invention, including each optional component described herein, to carry out the present invention. Coal was prepared and fed to a fluidized bed gasification reactor. Water, oxygen, and compressed carbon dioxide were also fed to the gasification reactor. The oxygen was first separated from the ambient air by an air separation unit. Oxygen separated from the air separation unit was fed to a sulfur recovery unit. The reaction within the gasification reactor produced coarse and fine ash, acid gas, and raw syngas at a rate of about 125.7 million standard cubic feet per day.

The coarse and fine ash from the gasification reactor was passed through an ash handing process and was then removed from the apparatus. About sixty eight percent of the total raw syngas from the gasification reactor was sent to a shift reactor.

The raw syngas from the gasification reactor was subjected to a shift reaction, low temperature gas cooling, and acid gas removal. About thirty two percent of the total raw syngas from the gasification reactor bypassed the shift reactor in this embodiment and was instead blended with about 41.5 million standard cubic feet per day of recycled syngas. All of the syngas (raw, shifted, and recycled) was then fed to an acid gas removal unit. The acid gas removal unit generated a sulfur rich gas, carbon dioxide, and syngas. The sulfur-rich gas was fed to the sulfur recovery unit. A portion of the hydrogen-rich permeate from a membrane separation unit was also fed to the sulfur recovery unit. A portion of the carbon dioxide from the acid gas removal unit was sent to a carbon dioxide compressor, and the remaining carbon dioxide was removed from the apparatus at a rate of about 54.7 million standard cubic feet per day. The sulfur recovery unit produced sulfur, which was removed from the apparatus, and a tail gas. The tail gas from the sulfur recovery unit was fed back to the acid gas removal unit. The syngas exiting the acid gas removal unit was fed to a methanol synthesis reactor at a rate of about 165.6 million standard cubic feet per day.

The carbon dioxide that was sent to the carbon dioxide compressor was compressed and fed back to the gasification reactor. The syngas from the acid gas removal unit was reacted in the methanol synthesis reactor at a pressure of about 5000 to 10000 KPa and a temperature of about 250° C. to produce a purge gas and crude methanol. A portion of the crude methanol was sent to storage, and the remaining portion was sent to a methanol distillation unit.

The purge gas from the methanol synthesis reactor was sent to a membrane separation unit, where the purge gas was divided into a relatively hydrogen-rich permeate stream and a relatively methane-rich non-permeate stream. A portion of the permeate stream was sent back to the methanol synthesis reactor.

The crude methanol was purified in the methanol distillation unit to generate at least substantially all grade AA methanol. The grade AA methanol was removed from the apparatus after passing through a storage and loading process at a rate of about 2210 short tons per day.

The non-permeate stream was fed to a steam methane reformer, which produced recycled syngas, flue gas, and steam. The flue gas and stream were removed from the apparatus. The recycled syngas, produced at a rate of about 41.5 million standard cubic feet per day, was blended with a portion of the raw syngas in a bypass stream around the shift reactor to increase the yield of methanol exiting the synthesis reactor.

While illustrative embodiments of the invention are disclosed herein, it will be appreciated that numerous modifications and other embodiments may be devised by those of ordinary skill in the art and the invention is not to be understood to be limited to such illustrated embodiments. Therefore, it will be understood that the appended claims are intended to cover all such expedient modifications and embodiments that come within the spirit and scope of the present invention, including those readily attainable by those of ordinary skill in the art from the disclosure set forth herein, or by routine experimentation therefrom. The specification and drawings are, accordingly, to be regarded in an illustrative sense rather than a restrictive sense.

What is claimed is:

1. A process for synthesizing a hydrocarbon comprising:
reacting a carbon-containing feedstock with an oxygen-containing gas stream having a greater molar ratio of oxygen than the feedstock to produce a first synthetic gas comprising hydrogen and carbon monoxide in a ratio;
reacting the first synthetic gas in a shift reaction zone to produce a shifted synthetic gas;
blending a recycled synthetic gas with the shifted synthetic gas to produce a second synthetic gas;
reacting the second synthetic gas in association with a catalyst in a synthesis reaction zone to produce one or more hydrocarbons and a purge gas;
passing the purge gas through a separation zone to produce a hydrogen-containing permeate stream having an increased hydrogen molar ratio compared to the purge gas and a methane-containing non-permeate stream having an increased methane molar ratio compared to the purge gas; and
reacting the methane-containing non-permeate stream in a reformation zone to produce the recycled synthetic gas.

2. The process of claim 1, wherein the blending of the recycled synthetic gas with the shifted synthetic gas increases the yield of the one or more hydrocarbons from the synthesis reaction zone.

3. The process of claim 1, which further comprises increasing the ratio of hydrogen to carbon monoxide in the synthesis reaction zone.

4. The process of claim 1, which further comprises separating ambient air in an air separation zone to increase the oxygen ratio in the oxygen-containing gas stream compared to air.

5. The process of claim 1, which further comprises removing one or more sulfur-containing compounds from the second synthetic gas.

6. The process of claim 5, which further comprises recovering an amount of sulfur from the one or more sulfur-containing compounds in a sulfur recovery zone.

7. The process of claim 1, which further comprises returning the hydrogen-containing permeate stream to the synthesis reaction zone.

8. The process of claim 1, wherein the carbon-containing feedstock comprises coal.

9. The process of claim 1, wherein the reformation zone comprises a steam methane reformer.

10. The process of claim 1, wherein the one or more hydrocarbons are selected to at least substantially comprise methanol.

11. A process for synthesizing methanol which comprises:
reacting a feedstock comprising coal and an oxygen-containing gas to produce a first synthetic gas comprising hydrogen and carbon monoxide in a ratio;
feeding at least a portion of the first synthetic gas to a shift reaction zone;
reacting the first synthetic gas in the shift reaction zone to produce a shifted synthetic gas;
blending a recycled synthetic gas with the shifted synthetic gas to produce a second synthetic gas;
reacting the second synthetic gas over a catalyst in a synthesis reaction zone to produce a product comprising methanol and a purge gas;
passing the purge gas through a separation zone to produce a hydrogen-containing permeate stream having an increased hydrogen molar ratio compared to the purge gas and a methane-containing non-permeate stream having an increased methane molar ratio compared to the purge gas; and
reacting the methane rich non-permeate stream in a steam methane reformation zone to produce the recycled synthetic gas.

12. The process of claim 11, which further comprises increasing the yield of the methanol from the synthesis reaction zone.

13. The process of claim 11, which further comprises increasing the ratio of hydrogen to carbon monoxide in the synthesis reaction zone.

14. The process of claim 11, which further comprises separating ambient air in an air separation zone to increase the oxygen ratio in the oxygen-containing gas stream compared to air.

15. The process of claim 11, which further comprises removing one or more sulfur-containing compounds from the second synthetic gas.

16. The process of claim 15, which further comprises recovering sulfur from the one or more sulfur-containing compounds in a sulfur recovery zone.

17. The process of claim 11, which further comprises returning the hydrogen-containing permeate stream to the synthesis reaction zone.

18. A hydrocarbon-generating apparatus comprising:
a first reactor adapted to react a carbon-containing feedstock and an oxygen-containing gas stream having a greater molar ratio of oxygen than the feedstock to produce a first synthetic gas comprising hydrogen and carbon monoxide in a ratio;
a shift reactor adapted to react the first synthetic gas to produce a shifted synthetic gas comprising carbon monoxide and hydrogen;
a synthesis reactor adapted to react a blend of the shifted synthetic gas and a recycled synthetic gas in association with a catalyst to produce one or more hydrocarbons and a purge gas;
a separation unit adapted to separate the purge gas into a hydrogen-containing permeate stream having an increased hydrogen molar ratio compared to the purge gas and a methane-containing non-permeate stream having an increased methane molar ratio compared to the purge gas; and
a reformation reactor adapted to react the methane-containing non-permeate stream to produce the recycled synthetic gas.

19. The hydrocarbon-generating apparatus of claim 18, wherein the shifted synthetic gas has an increased hydrogen to carbon dioxide ratio compared to the first synthetic gas.

20. The hydrocarbon-generating apparatus of claim 18, which further comprises an air separation unit adapted to separate oxygen from ambient air to increase the oxygen content in the oxygen-containing gas stream.

21. The hydrocarbon-generating apparatus of claim 18, which further comprises an acid gas removal unit adapted to remove CO₂ and one or more sulfur-containing compounds from the blend of the shifted synthetic gas and the recycled synthetic gas.

22. The hydrocarbon-generating apparatus of claim 21, which further comprises a sulfur recovery unit adapted to recover an amount of sulfur from the one or more sulfur-containing compounds.

23. The hydrocarbon-generating apparatus of claim 18, which further comprises a return stream that introduces the hydrogen-containing permeate stream into the synthesis reaction zone.
